(19) United States
(12) Patent Application Publication
(10) Pub. No.: US 2025/0073024 A1
(43) Pub. Date: Mar. 6, 2025

(54) EXPANDABLE FRAME FOR A PROSTHETIC HEART VALVE

(71) Applicant: EDWARDS LIFESCIENCES CORPORATION, Irvine, CA (US)

(72) Inventor: Tamir S. Levi, Zikhron Yaakov (IL)

(21) Appl. No.: 18/948,371

(22) Filed: Nov. 14, 2024

Related U.S. Application Data
(63) Continuation of application No. PCT/US2023/022218, filed on May 15, 2023.
(60) Provisional application No. 63/342,617, filed on May 16, 2022.

(51) Int. Cl. A61F 2/24

(52) U.S. Cl. CPC A61F 2/2418; A61F 2/2433; A61F 2250/0036

(57) ABSTRACT

Expandable frames for prosthetic heart valves and methods for implanting such prosthetic heart valves are disclosed. As one example, a prosthetic heart valve can include an annular frame comprising a plurality of interconnected struts arranged in a plurality of rows of struts, including a first row of first struts defining a first end of the frame, each first strut comprising an apex region disposed between two angled strut portions of the first strut. The frame is radially expandable from a radially compressed, delivery configuration to a radially expanded, deployed configuration, and each apex region is configured to bend radially outward from a first configuration to a second configuration where the apex region is bent radially outward at an angle relative to remaining struts in rows other than the first row of first struts, the apex region being in the second configuration when the frame is in the deployed configuration.

### CROSS-REFERENCE TO RELATED APPLICATIONS

This application is a continuation of PCT Application No. PCT/US2023/022218, filed May 15, 2023, which claims the benefit of U.S. Provisional Patent Application No. 63/342,617, filed May 16, 2022, both of which applications are incorporated by reference herein in their entireties.

### FIELD

The present disclosure relates to expandable prosthetic heart valves, and to methods and devices for implanting a prosthetic heart valve within an existing valvular structure.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Most expandable prosthetic heart valves comprise a radially expandable and compressible cylindrical metal frame or stent and prosthetic leaflets mounted inside the frame. In some examples, a prosthetic heart valve can be implanted within the aortic root, which includes the right and left coronary ostia and is defined between the native aortic valve annulus and the sinotubular junction (STJ). The prosthetic heart valve can be implanted either within the native aortic valve or within a previously implanted prosthetic heart valve (e.g., previously implanted via a valve-in-valve (ViV) procedure). However, during such implantations, there can be a risk of at least partially blocking the coronary ostia either by native aortic valve leaflets that are pushed sideways during expansion of the prosthetic heart valve, by prosthetic leaflets of a previously implanted prosthetic heart valve that are similarly pushed sideways during expansion of a new prosthetic heart valve during a ViV procedure, and/or by the overlapping frames of the two valves following the ViV procedure. As a result, access to the coronary arteries for later interventions (such as with a catheter) may be made more difficult.

Accordingly, a need exists for improved prosthetic heart valves and implantation methods that enable access to the coronary arteries (e.g., through a frame of the prosthetic heart valve) after prosthetic heart valve implantation and for future coronary artery interventions.

### SUMMARY

Described herein are prosthetic heart valves, delivery apparatuses, and methods for implanting prosthetic heart valves. In particular, described herein are examples of frames for prosthetic heart valves that have apices or apex regions that are configured to bend radially outward upon radial expansion of the prosthetic heart valves at a target implantation site. Prosthetic heart valves can include a frame and a leaflet assembly arranged on an interior of the frame. The frame can include a plurality of interconnected struts defining outflow apex regions (or apices) at an outflow end of the frame and inflow apex regions (or apices) at an inflow end of the frame. In some examples, the outflow apex regions have a straightened (or unbent) delivery configuration and are configured to bend radially outward upon radial expansion of the frame, into an outwardly bent configuration. The outwardly bent outflow apex regions can be configured to maintain the radially expanded prosthetic heart valve at a location in the aortic root that that maximizes a gap between an outflow end of the frame and outflow edges of the leaflets of the host valve in which the prosthetic heart valve is implanted (such as a previously implanted prosthetic heart valve), thereby maximizing a space for accessing the coronary ostia (through the frame) during subsequent coronary interventions (e.g., via a coronary access catheter). As such, the prosthetic heart valves, implantation methods, and delivery apparatuses disclosed herein can, among other things, overcome one or more of the deficiencies of typical prosthetic heart valves and their delivery apparatuses.

A prosthetic heart valve can comprise a frame and a valvular structure coupled to the frame. In addition to these components, a prosthetic heart valve can further comprise one or more of the components disclosed herein.

In some examples, the prosthetic heart valve can comprise a sealing member configured to reduce paravalvular leakage.

In some examples, the frame can comprise a plurality of interconnected struts, the plurality of interconnected struts arranged in a plurality of rows of struts, including a first row of first struts defining a first end of the frame, each first strut comprising an apex region.

In some examples, each apex region is disposed between two angled strut portions of the first strut.

In some examples, the frame is radially expandable from a radially compressed, delivery configuration to a radially expanded, deployed configuration.

In some examples, each apex region is configured to bend radially outward from a first configuration to a second configuration where the apex region is bent radially outward at an angle relative to remaining struts in rows other than the first row of first struts, the apex region being in the first configuration when the frame is in the delivery configuration and in the second configuration when the frame is in the deployed configuration.

In some examples, an end of each angled strut portion that is disposed away from the apex region and connects to a respective second strut of a second row of second struts has a neck region that has a narrowed thickness in a radial direction relative to a remainder of the first strut and the respective second strut of the second row of struts. A portion of the first strut including the apex region is configured to pivot and bend radially outward from the neck region of each angled strut portion relative to remaining struts in rows other than the first row of first struts, in response to a radially outwardly directed force applied to an inner surface of the frame.

In some examples, a prosthetic heart valve comprises an annular frame comprising a plurality of interconnected struts, the plurality of interconnected struts arranged in a plurality of rows of struts, including a first row of first struts defining a first end of the frame, each first strut comprising an apex region disposed between two angled strut portions of the first strut. The frame is radially expandable from a radially compressed, delivery configuration to a radially expanded, deployed configuration. Each apex region is configured to bend radially outward from a first configuration to a second configuration where the apex region is bent radially outward at an angle relative to remaining struts in rows other than the first row of first struts, the apex region being in the first configuration when the frame is in the delivery configuration and in the second configuration when the frame is in the deployed configuration.

In some examples, a prosthetic heart valve comprises a radially expandable and compressible annular frame comprising a plurality of interconnected struts defining a plurality of rows of cells arranged between an inflow end and an outflow end of the frame, the plurality of interconnected struts comprising a plurality of outflow struts defining the outflow end, where each outflow strut comprises two angled strut portions interconnected by an apex region. Each outflow strut is configured to bend radially outward in response to a radially outward force applied thereto during radial expansion of the frame and deployment of the prosthetic heart valve such that the apex region of each outflow strut extends radially outward at an angle relative to a longitudinal axis of remaining struts of the plurality of interconnected struts when the frame is in a radially expanded and deployed configuration, where the remaining struts are disposed between the outflow struts and the inflow end of the frame.

In some examples, a prosthetic heart valve comprises a radially expandable and compressible annular frame comprising a plurality of interconnected struts comprising a plurality of rows of struts including a first row of outflow struts defining an outflow end of the frame and a second row of struts connected to the first row of outflow struts, where each outflow strut comprises two angled strut portions interconnected by an apex region. An end of each angled strut portion that is disposed away from the apex region and connects to a respective strut of the second row of struts has a neck region that has a narrowed thickness in a radial direction relative to a remainder of the outflow strut and the respective strut of the second row of struts. A portion of the outflow strut including the apex region is configured to pivot and bend radially outward from the neck region of each angled strut portion relative to remaining struts in rows other than the first row of outflow struts, in response to a radially outwardly directed force applied to an inner surface of the frame.

In some examples, a prosthetic heart valve comprises one or more of the components recited in Examples 1-25, 55-63, and/or 74 below.

An assembly can comprise a prosthetic heart valve and a delivery apparatus.

In some examples, the assembly can comprise an inflatable balloon disposed at a distal end portion of the delivery apparatus and the prosthetic heart valve mounted around the balloon in a radially compressed configuration.

In some examples, the prosthetic heart valve comprises an annular frame.

In some examples, the annular frame comprises a plurality of interconnected struts, the plurality of interconnected struts arranged in a plurality of rows of struts, including a first row of outflow struts defining an outflow end of the frame, each outflow strut comprising an apex region disposed between two angled strut portions of the outflow strut.

In some examples, each apex region is configured to bend radially outward from a first configuration to a second configuration as a result of expansion of the balloon. In the second configuration the apex region is bent radially outward at an angle relative to remaining struts in rows other than the first row of first struts.

In some examples, an assembly comprises an inflatable balloon disposed at a distal end portion of a delivery apparatus and a prosthetic heart valve mounted around the balloon in a radially compressed configuration. The prosthetic heart valve comprises an annular frame comprising a plurality of interconnected struts, the plurality of interconnected struts arranged in a plurality of rows of struts, including a first row of outflow struts defining an outflow end of the frame, each outflow strut comprising an apex region disposed between two angled strut portions of the outflow strut, where each apex region is configured to bend radially outward from a first configuration to a second configuration as a result of expansion of the balloon. In the second configuration the apex region is bent radially outward at an angle relative to remaining struts in rows other than the first row of first struts.

In some examples, an assembly comprises one or more of the components recited in Examples 64-72 below.

A method can comprise radially expanding a prosthetic heart valve to a radially expanded configuration at a native valve annulus within an aortic root of a heart by expanding an inflatable balloon of a delivery apparatus around which the prosthetic heart valve is mounted, the prosthetic heart valve comprising a frame comprising a plurality of interconnected struts, the plurality of interconnected struts arranged in a plurality of circumferentially extending rows of struts including a first row of outflow struts defining an outflow end of the frame and a second row of inflow struts defining an inflow end of the frame.

In some examples, the method can comprise, in response to expanding the inflatable balloon, bending the outflow struts radially outward from struts of the frame to which the outflow struts are connected such that, after radially expanding the prosthetic heart valve to the radially expanded configuration, an apex region of each outflow strut is positioned radially outward relative to remaining struts of the frame that are disposed in rows of struts other than the first row of outflow struts.

In some examples, the method can include, bending the outflow struts radially outward such that, for at least a portion of the outflow struts, the apex region abuts a wall of the aortic root at or below a sinotubular junction.

In some examples, a method comprises advancing a prosthetic heart valve that is radially compressed around a distal end portion of a delivery apparatus to an implantation site using the delivery apparatus, where the distal end portion of the delivery apparatus includes an inflatable balloon. The method further comprises inflating the balloon of the delivery apparatus at the implantation site to radially expand and implant the prosthetic heart valve, where the inflating the balloon includes applying a radially outward pressure to a frame of the prosthetic heart valve with the balloon such that apex regions disposed at an outflow end of the frame are bent radially outward at an angle from remaining struts of the frame and relative to a longitudinal axis of the remaining struts that extends from an inflow end of the frame toward the outflow end. The frame comprises a plurality of interconnected struts arranged in a plurality of rows of struts, including a first row of outflow struts defining the outflow end of the frame and including the apex regions, and where the remaining struts are disposed in rows other than the first row of outflow struts.

In some examples, a method comprises radially expanding a prosthetic heart valve to a radially expanded configuration at a native valve annulus within an aortic root of a heart by expanding an inflatable balloon of a delivery apparatus around which the prosthetic heart valve is mounted, the prosthetic heart valve comprising a frame comprising a plurality of interconnected struts, the plurality of interconnected struts arranged in a plurality of circumferentially extending rows of struts including a first row of outflow struts defining an outflow end of the frame and a second row of inflow struts defining an inflow end of the frame. The method further comprises, in response to expanding the inflatable balloon, bending the outflow struts radially outward from struts of the frame to which the outflow struts are connected such that, after radially expanding the prosthetic heart valve to the radially expanded configuration, an apex region of each outflow strut is positioned radially outward relative to remaining struts of the frame that are disposed in rows of struts other than the first row of outflow struts and, for at least a portion of the outflow struts, the apex region abuts a wall of the aortic root at or below a sinotubular junction.

In some examples, a method comprises applying a radially outward pressure with a balloon of a delivery apparatus to an inner surface of a frame of a prosthetic heart valve to radially expand the prosthetic heart valve and, during the applying the radially outward pressure, bending outflow struts defining an outflow end of the frame radially outward from roots of the outflow struts that connect to axially extending struts of the frame such that apex regions of the outflow struts extend radially outward at an angle relative to a longitudinal axis of the axially extending struts. The method further comprises implanting the radially expanded prosthetic heart valve at a native valve annulus, within an aortic root of a heart, with at least a portion of the apex regions at the outflow end abutting a wall of the aortic root, below a sinotubular junction, while a remainder of the frame is spaced apart from the wall of the aortic root.

The above method(s) can be performed on a living animal or on a simulation, such as on a cadaver, cadaver heart, anthropomorphic ghost, simulator (e.g., with body parts, heart, tissue, etc. being simulated).

In some examples, a method comprises one or more of the features recited in Examples 26-54 and/or 73 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to one example.

FIG. 2 is a side view of a frame of the prosthetic heart valve of FIG. 1.

FIG. 3 is a side view of a portion of the frame of FIG. 2, showing the portion of the frame in a straightened (non-annular) state.

FIG. 4 is a side view of an exemplary delivery apparatus configured to deliver and implant a radially expandable prosthetic heart valve at an implantation site.

FIG. 5 is a side view of a portion of a frame for a prosthetic heart valve, according to one example, in a deployed configuration where apex regions at an outflow end of the frame are bent radially outward relative to remaining struts of the frame.

FIG. 6A is a cross-sectional view of the frame of FIG. 5 in a delivery configuration where the apex regions at the outflow end of the frame are unbent relative to remaining struts of the frame.

FIG. 6B is a cross-sectional view of the frame of FIG. 5 in the deployed configuration.

FIG. 7 shows the frame of FIG. 6A, in the delivery configuration, radially compressed around an inflatable balloon of an exemplary delivery apparatus.

FIG. 8 is a schematic illustrating an exemplary balloon having segments of varying compliance, including a reduced compliance segment that is configured to exert a radially outward force capable of bending the apex regions of the outflow struts of the frame of FIG. 5 radially outward, relative to a remainder of the frame, during radial expansion of the frame at an implantation site.

FIG. 9A is a cross-sectional view of a frame for a prosthetic heart valve, according to another example, in an unbent or delivery configuration where apex regions of outflow struts forming an outflow end of the frame are configured to bend radially outward from thinned portions of the outflow struts during radial expansion of the frame with a delivery apparatus.

FIG. 9B is a schematic illustrating another exemplary balloon bending the outflow struts of the frame of FIG. 9A radially outward from their thinned portions, relative to remaining struts of the frame, during inflation of the balloon at an implantation site.

FIG. 10 is a schematic depicting an exemplary positioning of a radially expanded prosthetic heart valve comprising the frame of FIG. 5 deployed within the aortic root of the aorta.

FIG. 11 is a flow chart of a method for implanting a prosthetic heart valve within an aortic root of a heart with radially outwardly bent apex regions or apices at an outflow end that hold the prosthetic heart valve in position at or below a sinotubular junction of the heart.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Overview of the Disclosed Technology

As introduced above, in some instances, prosthetic heart valves can be implanted at the aortic valve position, within the aortic root which includes the right and left coronary ostia and is defined between the native aortic valve annulus and the STJ. As the initial age of patients requiring a prosthetic heart valve decreases, the occurrence of subsequent interventions in those patients increases, such as valve-in-valve procedures (or ViV procedures) where a second (or third) prosthetic heart valve is implanted within a previously implanted prosthetic heart valve. Following such ViV procedures, or after implantation of a prosthetic heart valve within the native aortic valve, further interventions requiring access to the coronary arteries (e.g., via a catheter) may be necessary. Thus, the inventors herein have recognized that it is important to maximize open spaces through the frame (at an outflow end of the frame) of the implanted prosthetic heart valve for accessing the coronary arteries. Such access openings or windows through the frame can be defined both between the outflow end of the prosthetic valve and the outflow edges of the host valve leaflets (which can be leaflets of the native aortic valve and/or the previously implanted prosthetic heart valve) and between adjacent struts of the newly implanted prosthetic heart valve and a previously implanted prosthetic heart valve.

For example, in some instances, upon implantation of the prosthetic heart valve within a host aortic valve (which can be the native aortic valve or a previously implanted prosthetic heart valve within the native aortic valve), leaflets of the host valve can be pushed sideways during radial expansion of the (new) prosthetic heart valve. As a result, the host leaflets can be pushed toward the coronary ostia and at least partially block access to the coronary arteries (e.g., by reducing a size of the openings through the frame for a coronary access device to pass through). In some instances, the opening or window for accessing the coronary arteries through the frame of the prosthetic heart valve can be defined between the host leaflets (e.g., of the native aortic valve or previously implanted prosthetic valve) and the STJ or sinus ceiling. Since the sinus ceiling is angled and tapers outward from the STJ to the sinus proximate to the coronary ostia, the access window through the frame of the prosthetic heart valve can be reduced due to the outflow apices (e.g., a longitudinal axis of the outflow apices) of the prosthetic valve being non-perpendicular to the angled wall of the sinus ceiling. Further, if the prosthetic heart valve is implanted at or above the STJ (or sinus ceiling), the access window to the coronary arteries through the frame of the implanted prosthetic heart valve can also be reduced and subsequent intervention can be made more difficult.

Described herein are various examples of frames for prosthetic heart valves that include a plurality of apex regions or apices at an outflow end of the frame that are bendable radially outward during radial expansion of the frame (e.g., via delivery apparatus) and that can retain the outwardly bent configuration after deployment at the implantation site. As a result, the outwardly bent apex regions can interface with the angled sinus ceiling, at or below the STJ, at an angle (e.g., approximately 90 degrees) that maximizes the distance between the outflow end of the frame and the host valve leaflets, thereby maximizing a size of the windows for a device, such as a catheter, to access the coronary arteries during a later interventional procedure. Additionally, by abutting the angled sinus ceiling, the outwardly bent apex regions of the prosthetic heart valve can be better held in place within the aortic root and migration of the prosthetic heart valve into the ascending aorta can be avoided.

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

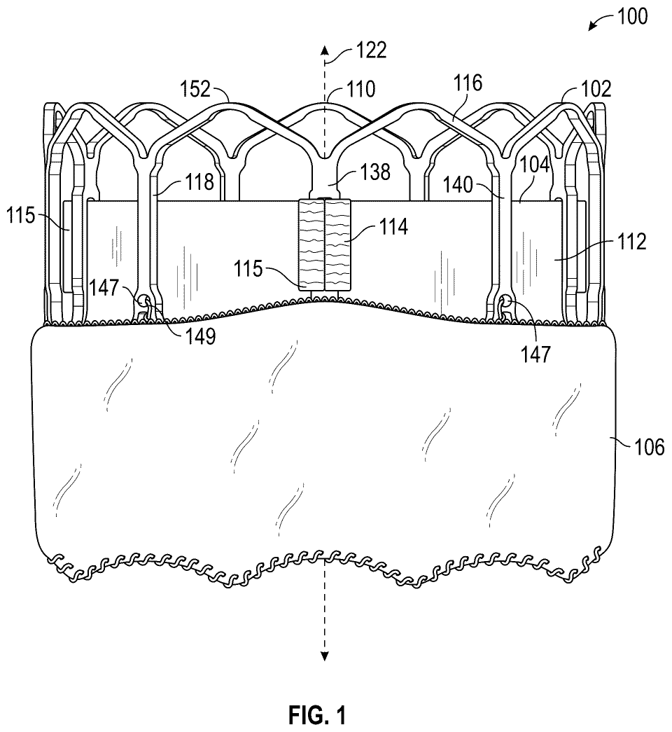

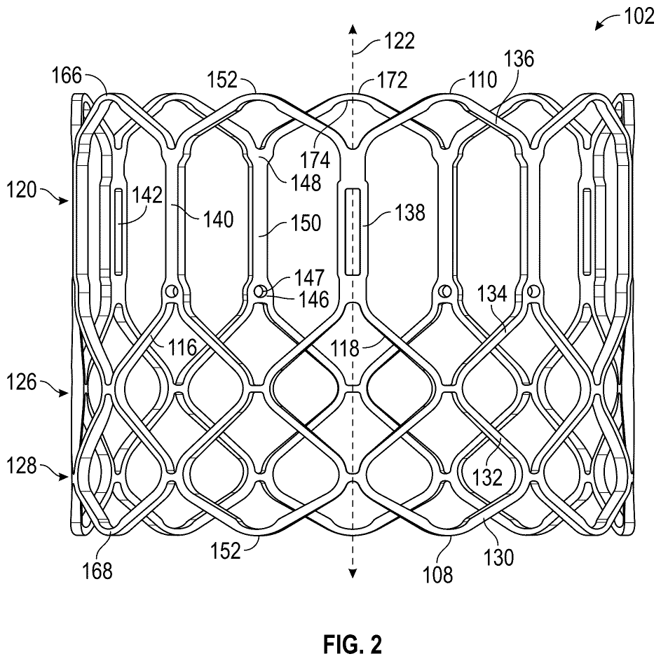

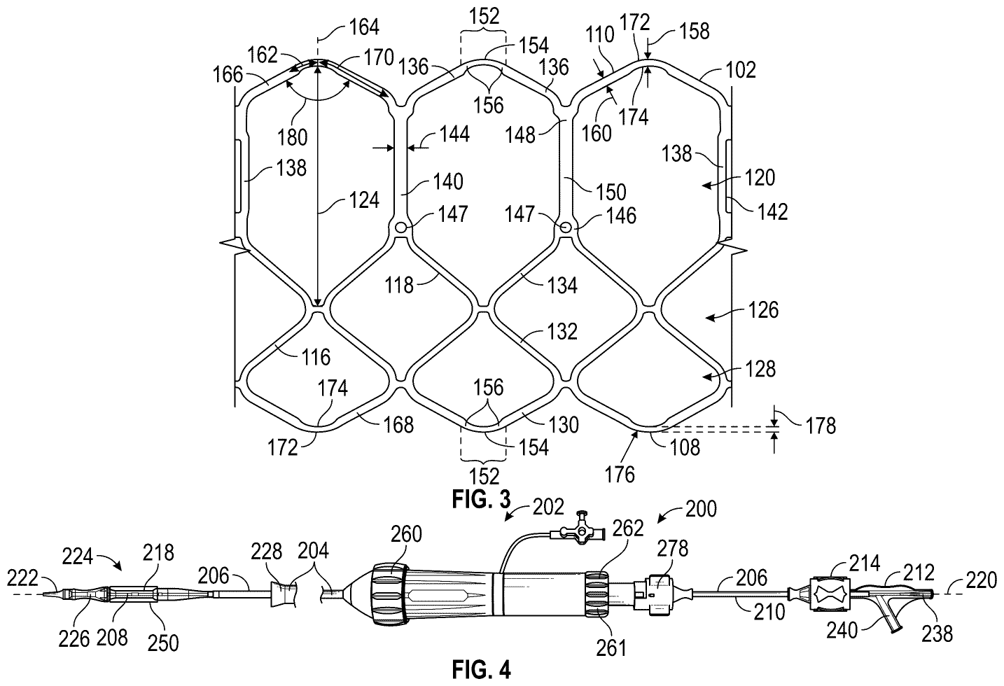

FIG. 1 illustrates an exemplary prosthetic device (e.g., prosthetic heart valve) comprising a frame, leaflets secured on an inside of the frame, and an outer skirt disposed around an outer surface of the frame. In some examples, the frame can comprise a plurality of interconnected and angled struts and apex regions that extend and/or curve between the angled struts at an inflow end and outflow end of the frame, as shown in FIGS. 2 and 3. The prosthetic device can be advanced through a patient's vasculature, such as to a native heart valve, by a delivery apparatus, such as the exemplary delivery apparatus shown in FIG. 4. In some examples, the deliver apparatus can include an expandable balloon configured to inflate and radially expand the prosthetic heart valve at the implantation site.

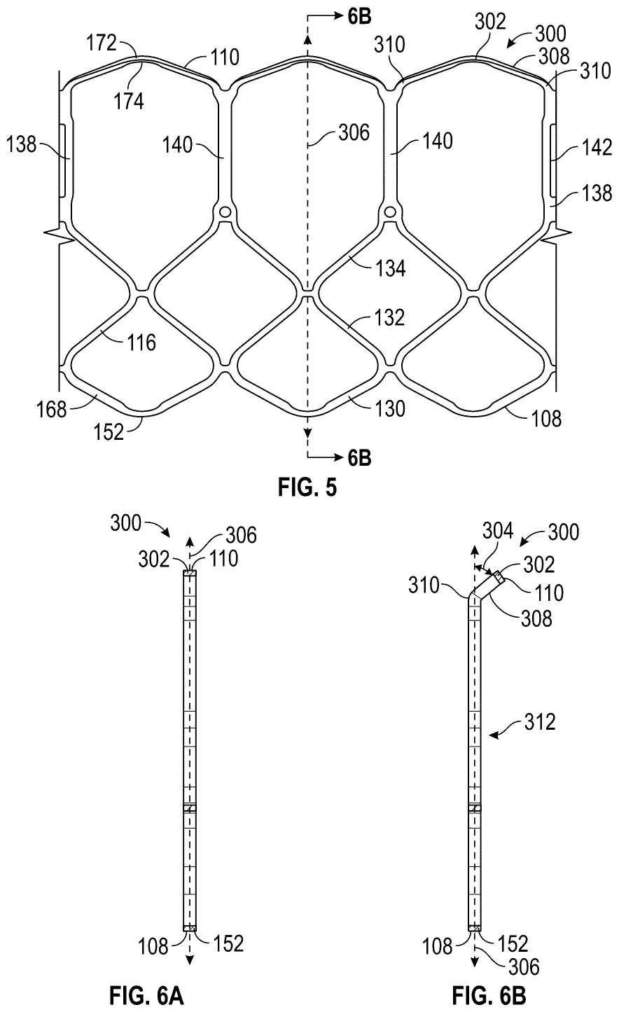

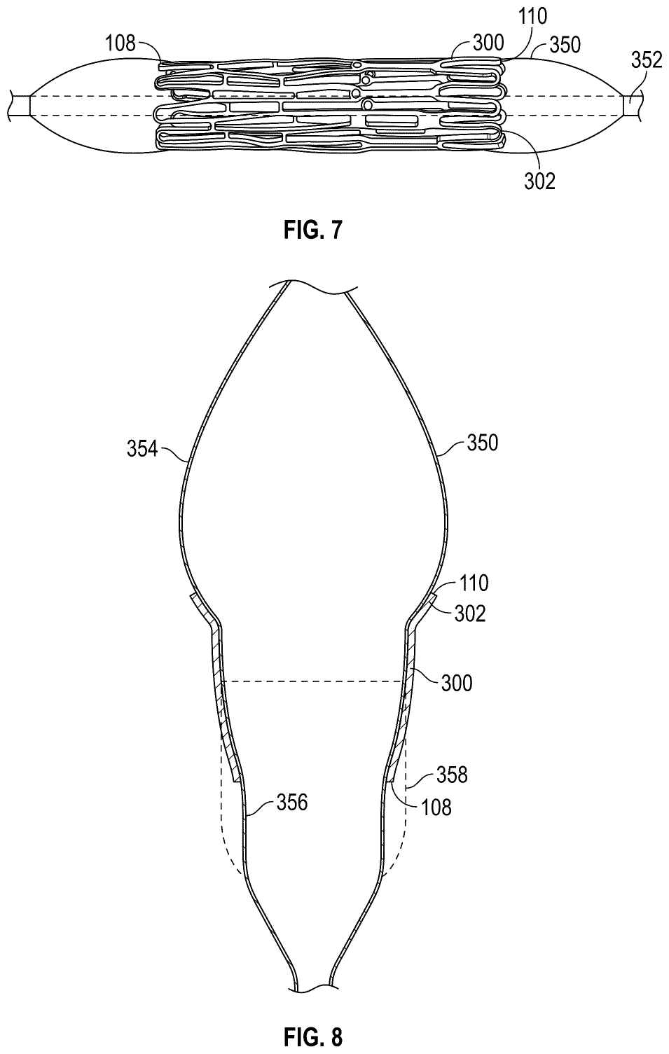

In some examples, a frame for a prosthetic heart valve, such as the frame shown in FIGS. 5-7, can comprise apex regions at its outflow end that are configured to bend radially outward upon radial expansion of the prosthetic heart valve with a delivery apparatus from a relatively straight or unbent configuration (its delivery configuration, as shown in FIGS. 6A and 7) to an outwardly bent configuration (its deployed configuration, as shown in FIGS. 5 and 6B).

In some instances, the outward bending of the apex regions at the outflow end of the prosthetic heart valve can be achieved by the balloon of the delivery apparatus. For example, as shown in FIG. 8, a balloon of varying compliance can be used to apply increased radially outward pressure to the apex regions at the outflow end of the frame, thereby bending the apex regions radially outward relative to a remainder of the frame during expansion of the balloon.

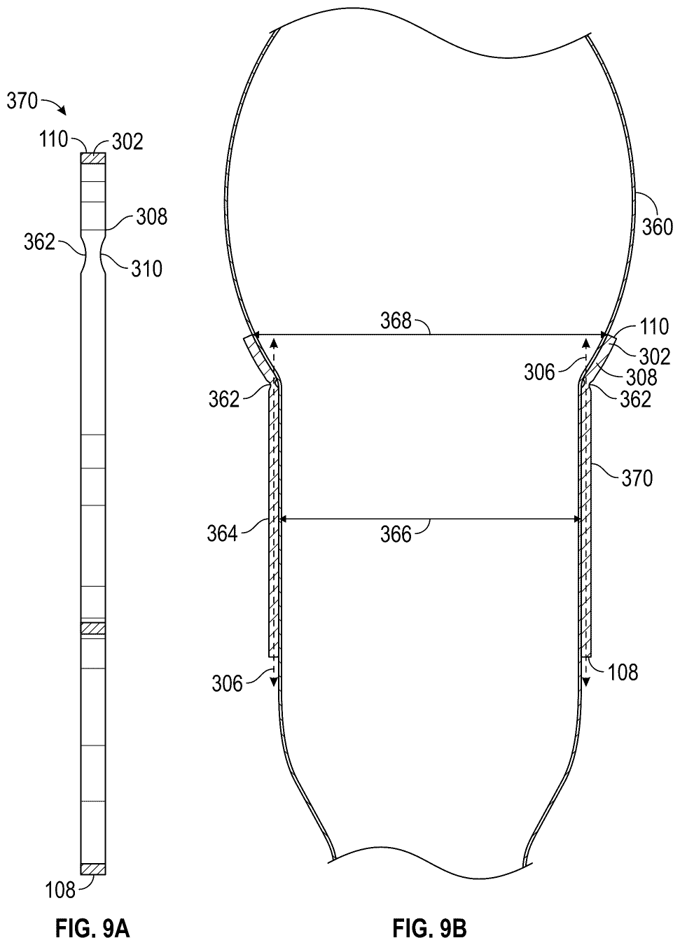

In alternate instances, the outward bending of the apex regions at the outflow end of the prosthetic heart valve can be achieved by thinner walls at a root of the outflow struts defining the outflow end of the frame (and the apex regions at the outflow end) (FIG. 9A). For example, as shown in FIGS. 9A and 9B, the roots of the outflow struts can be narrower in a radial direction relative to the struts to which they are connected and a remainder of the outflow struts, thereby causing the apex regions of the outflow struts to pivot from the narrower roots and bend radially outward in response to radial outward pressure from the balloon during expansion of the balloon (as shown in FIG. 9B).

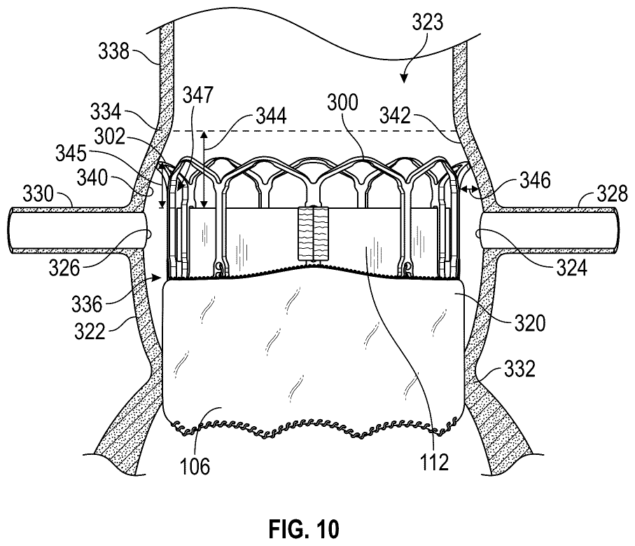

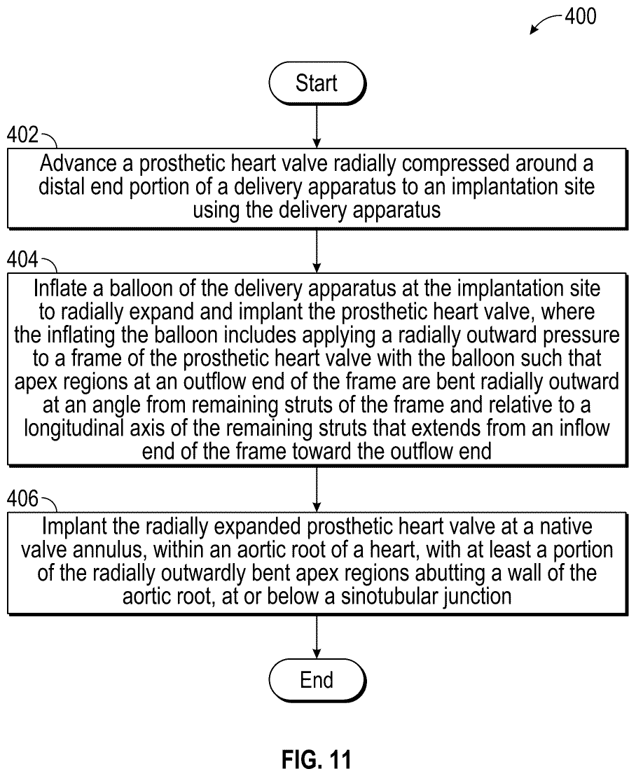

Thus, as shown in FIG. 10 and described in the method presented in FIG. 11, the prosthetic heart valve can be implanted within the aortic root and held below (or at) the STJ and sinus ceiling by the radially outwardly bent apex regions at the outflow end of the prosthetic heart valve. As a result of the outwardly bent apex regions abutting the angled sinus ceiling, openings for accessing the coronary arteries through the frame of the implanted prosthetic heart valve can be maximized and the prosthetic heart valve can be better held in place within the aortic root (e.g., without migrating into the ascending aorta).

### Examples of the Disclosed Technology

FIG. 1 shows a prosthetic heart valve 100 (prosthetic valve), according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic heart valve 100 can include a stent or frame 102, a valvular structure 104, and a perivalvular outer sealing member or outer skirt 106. The prosthetic heart valve 100 (and the frame 102) can have an inflow end 108 and an outflow end 110. The valvular structure 104 can be disposed on an interior of the frame 102 while the outer skirt 106 is disposed around an outer surface of the frame 102.

The valvular structure 104 can comprise a plurality of leaflets 112 (e.g., three leaflets, as shown in FIG. 1), collectively forming a leaflet structure, which can be arranged to collapse in a tricuspid arrangement. The leaflets 112 can be secured to one another at their adjacent sides (e.g., commissure tabs) to form commissures 114 of the valvular structure 104. For example, each leaflet 112 can comprise opposing commissure tabs disposed on opposite sides of the leaflet 112 and a cusp edge portion extending between the opposing commissure tabs. The cusp edge portion of the leaflets 112 can have an undulating, curved scalloped shape, and can be secured directly to the frame 102 (e.g., by sutures). However, in alternate examples, the cusp edge portion of the leaflets 112 can be secured to an inner skirt which is then secured to the frame 102. In some examples, the leaflets 112 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

In some examples, the outer skirt 106 can be an annular skirt. In some instances, the outer skirt 106 can comprise one or more skirt portions that are connected together and/or individually connected to the frame 102. The outer skirt 106 can comprise a fabric or polymeric material, such as ePTFE, PTFE, PET, TPU, UHMWPE, PEEK, PE, etc. In some instances, instead of having a relatively straight upper edge portion, as shown in FIG. 1, the outer skirt 106 can have an undulating upper edge portion that extends along and is secured to the angled struts 134. Examples of such outer skirts, as well as various other outer skirts, that can be used with the frame 102 can be found in the provisional patent application under Edwards' attorney docket No. 12131US01, which is incorporated by reference herein.

The frame 102 can be radially compressible and expandable between a radially compressed configuration and a radially expanded configuration (the expanded configuration is shown in FIG. 1). The frame 102 is shown alone in FIG. 2 and a portion of the frame 102 in a straightened (non-annular) configuration is shown in FIG. 3.

The frame 102 can be made of any of various suitable plastically-expandable materials (e.g., stainless steel, etc.) or self-expanding materials (e.g., Nitinol). When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (e.g., the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As shown in FIGS. 2 and 3, the frame 102 can comprise a plurality of interconnected struts 116 which form multiple rows of open cells 118 between the outflow end 110 and the inflow end 108 of the frame 102. In some examples, as shown in FIGS. 2 and 3, the frame 102 can comprise three rows of cells 118 with a first (upper in the orientation shown in FIGS. 2 and 3) row of cells 120 disposed at the outflow end 110. The first row of cells 120 comprises cells 118 that are elongated in an axial direction (relative to a central longitudinal axis 122 of the frame 102), as compared to cells 118 in the remaining rows of cells. For example, the cells 118 of the first row of cells 120 can have a longer axial length 124 (FIG. 3) than cells 118 in the remaining rows of cells, which can include a second row of cells 126 and a third row of cells 128, the third row of cells 128 disposed at the inflow end 108 and the second row of cells 126 disposed between the first row of cells 120 and the third row of cells 128.

In some examples, as shown in FIG. 2, each row of cells comprises nine cells 118. Thus, in such examples, the frame 102 can be referred to as a nine-cell frame.

In alternate examples, the frame 102 can comprise more than three rows of cells (e.g., four or five) and/or more or less than nine cells per row. In some examples, the cells 118 in the first row of cells 120 may not be elongated compared to cells 118 in the remaining rows of cells of the frame 102 (the second row of cells 126 and the third row of cells 128).

The interconnected struts 116 can include a plurality of angled struts 130, 132, 134, and 136 arranged in a plurality of rows of circumferentially extending rows of angled struts, with the rows being arrayed along the length of the frame 102 between the outflow end 110 and the inflow end 108. For example, the frame 102 can comprise a first row of angled struts 130 arranged end-to-end and extending circumferentially at the inflow end 108 of the frame; a second row of circumferentially extending, angled struts 132; a third row of circumferentially extending, angled struts 134; and a fourth row of circumferentially extending, angled struts 136 at the outflow end 110 of the frame 102. The fourth row of angled struts 136 can be connected to the third row of angled struts 134 by a plurality of axially extending window struts 138 (or window strut portions) and a plurality of axial (e.g., axially extending) struts 140. The axially extending window struts 138 (which can also be referred to as axial struts that include a commissure window) define commissure windows (e.g., open windows) 142 that are spaced apart from one another around the frame 102, in a circumferential direction, and which are adapted to receive a pair of commissure tabs of a pair of adjacent leaflets 112 arranged into a commissure (e.g., commissure 114 shown in FIG. 1). In some examples, the commissure windows 142 and/or the axially extending window struts 138 defining the commissure windows 142 can be referred to herein as commissure features or commissure supports, each commissure feature or support configured to receive and/or be secured to a pair of commissure tabs of a pair of adjacent leaflets.

One or more (e.g., two, as shown in FIGS. 2 and 3) axial struts 140 can be positioned between, in the circumferential direction, two commissure windows 142 formed by the window struts 138. Since the frame 102 can include fewer cells per row (e.g., nine) and fewer axial struts 140 between each commissure window 142, as compared to some more traditional prosthetic heart valves, each cell 118 can have an increased width (in the circumferential direction), thereby providing a larger opening for blood flow and/or coronary access.

Each axial strut 140 and each window strut 138 extends from a location defined by the convergence of the lower ends (e.g., ends arranged inward of and farthest away from the outflow end 110) of two angled struts 136 (which can also be referred to as an upper strut junction or upper elongated strut junction) to another location defined by the convergence of the upper ends (e.g., ends arranged closer to the outflow end 110) of two angled struts 134 (which can also be referred to as a lower strut junction or lower elongate strut junction). Each axial strut 140 and each window strut 138 forms an axial side of two adjacent cells of the first row of cells 120.

In some examples, as shown in FIG. 3, each axial strut 140 can have a width 144 (FIG. 3) that is larger than a width of the angled struts 130, 132, 134, and 136. As used herein, a “width” of a strut is measured between opposing locations on opposing surfaces of a strut that extend between the radially facing inner and outer surfaces of the strut (relative to the central longitudinal axis 122 of the frame 102). A “thickness” of a strut is measured between opposing locations on the radially facing inner and outer surfaces of a strut and is perpendicular to the width of the strut. In some examples, the width 144 of the axial struts 140 is 50-200%, 75-150%, or at least 100% larger than (e.g., double) the width of the angled struts of the frame 102.

By providing the axial struts 140 with the width 144 that is greater than the width of other, angled struts of the frame 102, a larger contact area is provided for when the leaflets 112 contact the wider axial struts 140 during systole, thereby distributing the stress and reducing the extent to which the leaflets 112 may fold over the axial struts 140, radially outward through the cells 118. As a result, a long-term durability of the leaflets 112 can be increased.

Since the cells 118 of the frame 102 can have a relatively large width compared to alternate prosthetic valves that have more than nine cells per row (as introduced above), the wider axial struts 140 can be more easily incorporated into the frame 102, without sacrificing open space for blood flow and/or coronary access.

Commissure tabs 115 of adjacent leaflets 112 can be secured together to form commissures 114 (FIG. 1). Each commissure 114 of the prosthetic heart valve 100 comprises two commissure tabs 115 paired together, one from each of two adjacent leaflets 112, and extending through a commissure window 142 of the frame 102. Each commissure 114 can be secured to the window struts 138 forming the commissure window 142.

The cusp edge portion (e.g., scallop edge) of each leaflet 112 can be secured to the frame 102 via one or more fasteners (e.g., sutures). In some examples, the cusp edge portion of each leaflet 112 can be secured directly to the struts of the frame 102 (e.g., angled struts 130, 132, and 134). For example, the cusp edge portions of the leaflets 112 can be sutured to the angled struts 130, 132, and 134 that generally follow the contour of the cusp edge portions of the leaflets 112.

In some examples, the cusp edge portion of the leaflets 112 can be secured to an inner skirt and the inner skirt can then be secured directly to the frame 102.

Various methods for securing the leaflets 112 to a frame, such as the frame 102, are disclosed in U.S. provisional patent applications 63/278,922, filed Nov. 12, 2021, and 63/300,302, filed Jan. 18, 2022, both of which are incorporated by reference herein.

As shown in FIGS. 2 and 3, in some examples, one or more of or each of the axial struts 140 can comprise an inflow end portion 146 (e.g., an end portion that is closest to the inflow end 108) and an outflow end portion 148 that are widened relative to a middle portion 150 of the axial strut 140 (which can be defined by the width 144). In some instances, the inflow end portion 146 of the axial strut 140 can comprise an aperture 147. The apertures 147 can be configured to receive fasteners (e.g., sutures) for attaching soft components of the prosthetic heart valve 100 to the frame 102. For example, in some instances, the outer skirt 106 can be positioned around the outer surface of the frame 102 and an upper or outflow edge portion of the outer skirt 106 can be secured to the apertures 147 by fasteners 149 (e.g., sutures), as shown in FIG. 1.

The frame 102 can further comprise a plurality of apex regions 152 formed at the inflow end 108 and the outflow end 110, each apex region 152 extending and forming a junction between two angled struts 130 at the inflow end 108 or two angled struts 136 at the outflow end 110. As such, the apex regions 152 are spaced apart from one another, in a circumferential direction at the inflow end 108 and the outflow end 110.

Each apex region 152 can comprise an apex 154 (the highest or most outward extending, in an axial direction, point) and two thinned (or narrowed) strut portions 156, one thinned strut portion 156 extending from either side of the apex 154 to a corresponding, wider, angled strut 136 (at the outflow end 110) or angled strut 130 (at the inflow end 108) (FIG. 3). In this way, each of the apex regions 152 at the outflow end 110 can form a narrowed transition region between and relative to the two angled struts 136 extending from the corresponding apex region 152 and each of the apex regions 152 at the inflow end 108 can form a narrowed transition region between and relative to the two angled struts 130 extending from the corresponding apex region 152.

The thinned strut portions 156 of the apex regions 152 can have a width 158 that is smaller than a width 160 of the angled struts 130 or 136 (FIG. 3). In some examples, the width 158 can be a uniform width (e.g., along an entire length of the strut portion 156). In some examples, the width 158 of the thinned strut portions 156 can be from about 0.06-0.15 mm smaller than the width 160 of the angled struts 130 and/or 136.

The thinned strut portions 156 of the apex regions 152 can have a first length 162 (FIG. 3). In some examples, the first length 162 is in a range of 0.8-1.4 mm, 0.9-1.2 mm, 0.95-1.05 mm, or about 1.0 mm (e.g., ±0.03 mm). In alternate examples, the first length 162 is in a range of 0.3-0.7 mm, 0.4-0.6 mm, 0.45-0.55 mm, or about 0.5 mm (e.g., ±0.03 mm).

Thus, each outflow apex region 152 can include two thinned strut portions 156 having the first length 162, each extending from the apex 154, outward relative to a central longitudinal axis 164 of the cells 118. Thus, a total length of the apex region 152 can be two times the first length 162.

Each apex region 152 and two corresponding angled struts 136 at the outflow end 110 can form an outflow strut 166 and each apex region 152 and two corresponding angled struts 130 at the inflow end 108 can form an inflow strut 168.

Each outflow strut 166 and inflow strut 168 can have a length that includes an apex region 152 and the two angled struts 136 or 130 (or strut portions), respectively, on either side of the apex region 152. One half the total length of each outflow strut 166 and inflow strut 168 is shown in FIG. 3 as length 170, which extends from an end of one angled strut 136 or 130 to the central longitudinal axis 164. Thus, the length of each outflow strut 166 and inflow strut 168 is two times length 170. In some examples, the length 170 for half of each inflow strut 168 can be different than the length 170 for half of each outflow strut 166.

In some instances, the length of each thinned strut portion 156 can be at least 25% of the length 170 of the corresponding half outflow strut 166 or inflow strut 168. Said another way, the length of each apex region 152 (a total length being two times the first length 162) can be at least 25% of the total length (two times length 170) of the outflow strut 166 or inflow strut 168. In some examples, the length of each apex region 152 can be more than 25% of the total length of the corresponding outflow strut 166 or inflow strut 168, such as 25-35%.

In some examples, each apex region 152 can comprise a curved, axially facing outer surface 172 and an arcuate or curved, axially facing inner depression 174 which forms the thinned strut portions 156. For example, the curved inner depression 174 can depress toward the curved outer surface 172 from an inner surface of the angled strut portions 156, thereby forming the smaller width thinned strut portions 156. Thus, the curved inner depressions 174 can be formed on a cell side of the apex region 152 (e.g., as opposed to the outside of the apex region 152).

In some examples, the curved outer surface 172 of each apex region 152 can form a single, continuous curve from one angled strut portion 156 on a first side of the apex region 152 to another angled strut portion 156 on an opposite, second side of the apex region 152.

Each apex region 152 can have a radius of curvature 176, along the curved outer surface 172 (e.g., in some instances, along an entirety or an entire length of the curved outer surface 172) (FIG. 3). In some instances, the radius of curvature 176 at the apex 154 and/or along the entire curved outer surface 172 of the apex region 152 can be greater than 1 mm. In some instances, the radius of curvature 176 can be in a range of 1-20 mm, 3-16 mm, or 8-14 mm. In some instances, the radius of curvature 176 can be greater than 10 mm. The radius of curvature 176 can be dependent on (and thus change due to changes in) the width 158 (e.g., the amount of reduction in width from the angled struts 130 or 136) and the first length 162 of the thinned strut portions 156.

Further, a height (an axial height) 178 of the apex regions 152, which can be defined in the axial direction from an outer surface of the two angled struts 130 or 136 to the curved outer surface 172 of the apex region 152 at the apex 414, can be the width 158 of the thinned strut portions 156 (FIG. 3). In this way, the height 178 of the apex regions 152 can be relatively small and not add much to the overall axial height of the radially expanded frame 102. Thus, the leaflets 112 secured to the frame 102 (FIG. 1) can be disposed close to the inflow end 108, thereby leaving a lar If ger open space at the outflow end 110 of the frame 102 that is not blocked by the leaflets 112.

In some examples, each of the apex region 152 can form an angle 180 between the two angled struts 130 or 136 extending from either side of the corresponding apex region 152 (FIG. 3). In some instances, the angle 180 can be in a range of 120 (not inclusive) to 140 degrees (e.g., such that the angle 180 is greater than 120 degrees and less than or equal to 140 degrees).

Additional details and examples of frames for prosthetic heart valves that include apex regions can be found in U.S. Provisional Patent Application Nos. 63/178,416, filed Apr. 22, 2021, 63/194,830, filed May 28, 2021, and 63/279,096, filed Nov. 13, 2021, all of which are incorporated by reference herein.

FIG. 4 shows a delivery apparatus 200, according to an example, that can be used to implant an expandable prosthetic heart valve (e.g., the prosthetic heart valve 100 of FIG. 1 and/or any of the other prosthetic heart valves described herein). In some examples, the delivery apparatus 200 is specifically adapted for use in introducing a prosthetic valve into a heart.

The delivery apparatus 200 in the illustrated example of FIG. 4 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (e.g., move) longitudinally, along a central longitudinal axis 220 of the delivery apparatus 200, relative to one another to facilitate delivery and positioning of a prosthetic valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (e.g., inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft 208 can extend distally beyond the distal end portion of the intermediate shaft 206.

The balloon 218 can be coupled to the distal end portion of the intermediate shaft 206.

In some examples, a distal end of the balloon 218 can be coupled to a distal end of the delivery apparatus 200, such as to a nose cone 222 (as shown in FIG. 4), or to an alternate component at the distal end of the delivery apparatus 200 (e.g., a distal shoulder). An intermediate portion of the balloon 218 can overlay a valve mounting portion 224 of a distal end portion of the delivery apparatus 200 and a distal end portion of the balloon 218 can overly a distal shoulder 226 of the delivery apparatus 200. The valve mounting portion 224 and the intermediate portion of the balloon 218 can be configured to receive a prosthetic heart valve in a radially compressed state. For example, as shown schematically in FIG. 4, a prosthetic heart valve 250 (which can be one of the prosthetic valves described herein) can be mounted around the balloon 218, at the valve mounting portion 224 of the delivery apparatus 200.

The balloon shoulder assembly, including the distal shoulder 226, is configured to maintain the prosthetic heart valve 250 (or other medical device) at a fixed position on the balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic valve 250 is mounted in the radially compressed state on the valve mounting portion 224 (as shown in FIG. 4) and during delivery of the prosthetic valve to the target implantation site. As such, the distal tip portion 228 can be configured to resist movement of the prosthetic valve 250 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the prosthetic valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (e.g., for fine positioning at the implantation site). Further details on the delivery apparatus 200 can be found in PCT Application No. PCT/US2021/047056, which is incorporated by reference herein.

In some examples, the frame 102 or another frame for a prosthetic heart valve can have apex regions (or apices) that are configured to bend radially outward during radial expansion of the prosthetic heart valve. In some examples, the apex regions that are configured to bend radially outward from an outflow end of the frame. In alternative examples, the apex regions that are configured to bend radially outward form an inflow end of the frame. In still other examples, the apex regions at both the inflow end and the outflow end of the frame can be configured to bend radially outward relative to an intermediate portion of the frame (e.g., struts of the frame disposed between the outflow and inflow struts of the frame).

For example, FIG. 5 illustrates a portion of a frame 300 for a prosthetic heart valve in a straightened (non-annular) configuration (for the purpose of illustration) which includes radially outwardly bent or extending apex regions 302 at the outflow end 110 of the frame 300. In some examples, the frame 300 can be used in lieu of the frame 102 in the prosthetic heart valve 100 of FIG. 1.

The frame 300 can be similar to the frame 102, except it includes apex regions 302 at the outflow end 110 (and thus can be referred to as outflow apex regions 302) that are configured to bend radially outward from an initial, straightened configuration (an unbent configuration) to a radially outwardly bent configuration (a deployed configuration). The frame 300 can be in the straightened configuration following manufacturing (e.g., when partially or fully radially expanded, but prior to being radially compressed onto and deployed by a delivery apparatus) and/or when mounted on a delivery apparatus in a radially compressed configuration (as shown in FIG. 7 and also referred to herein as a delivery configuration).

In some instances, the frame 102 of FIGS. 2 and 3 can represent the frame 300 in the straightened or unbent configuration. FIG. 6A shows an exemplary cross-section of the frame 300 when in the straightened or unbent configuration. Additionally, FIG. 7 shows the frame 300 radially compressed around an inflatable balloon 350 of a delivery apparatus 352 (which, in some instances, can be the delivery apparatus 200 of FIG. 4 or another delivery apparatus comprising an inflatable a balloon). As shown in FIG. 7, the frame 300 is radially compressed around the balloon 350 and the apex regions 302 at the outflow end 110 are in the initial, straightened configuration.

As described in further detail below with reference to FIGS. 8-9B, when the prosthetic heart valve, which is radially compressed onto the distal end portion of the delivery apparatus, reaches the target implantation site (e.g., the native aortic annulus, within the aortic root), the delivery apparatus can be used to inflate the balloon 350 and radially expand the prosthetic heart valve within the native heart valve or a previously implanted prosthetic heart valve. As the balloon 350 expands, the apex regions 302 can be bent radially outward from a remainder of the frame 300 in response to a radially outward force applied thereto, such as by balloon inflation, thereby moving the apex regions 302 into the deployed configuration shown in FIG. 5 and the cross-sectional view of FIG. 6B.

As shown in FIGS. 5 and 6A, the frame 300 can be the same as the frame 102, except for the radially outwardly bendable outflow apex regions 302. Thus, similar components to the frame 102 have been labeled similarly in FIGS. 5-7. For example, the frame 300 can comprise apex regions 152 at its inflow end 108 that remain relatively unbent both before (FIGS. 6A and 7) and after deployment with the delivery apparatus (FIGS. 5 and 6B). In some instances, the apex regions 302 can have the same overall geometry as the apex regions 152, except for their outwardly bent configuration following deployment with the delivery apparatus (as shown in FIGS. 5 and 6B).

In alternate examples, the apex regions 152 at the inflow end 108 can also be configured to bend radially outward upon radial expansion of the frame 300 with a delivery apparatus.

In still other examples, the outwardly bendable outflow apex regions 302 can be used in various frame types, including those with differently shaped apex regions or apices. For example, in some instances, the frame can include inflow or outflow struts having apices with a more pointed profile that are configured to bend radially outward relative to remaining struts of the frame.

However, as explained further below, the apex regions 302 with the curved and reduced height configuration (as described above with reference to FIGS. 2 and 3 for apex regions 152) can be advantageous over apices that are more pointed or that have harsher angles. For example, when in the radially outwardly bent and deployed configuration (FIGS. 5 and 6B), the apex regions 302 provide a curved outward facing surface for interfacing with the native tissue, thereby providing a more atraumatic surface against the tissue.

As shown in FIGS. 5 and 6B, in the bent and deployed configuration, the apex regions 302 can be bent radially outward (relative to a central longitudinal axis of the frame, such as the central longitudinal axis 122 shown in FIG. 2) at an angle 304 (FIG. 6B). The angle 304 is defined between the apex region 302 (or bent portion of the corresponding outflow strut 308) and a vertical or longitudinal axis 306 of the struts (unbent struts, such as the axially extending struts 140 and 138) of the frame 300 which can extend between the inflow end 108 and the outflow end 110 (or along an intermediate portion 312 of the frame 300 defined between outflow and inflow struts of the frame 300, FIG. 6B).

Each apex region 302 can be part of an outflow strut 308 of the frame 300 (which can be similar to the outflow struts 166) and each outflow strut 308 can connect to two axial struts 140 or an axial strut 140 and an axially extending window strut 138 at roots 310 (or ends) of the outflow strut 308. In some examples, the outflow strut 308, including the apex region 302, can bend radially outward from the roots 310 of the outflow strut 308. In alternate examples, the apex region 302 can bend radially outward from a portion of the outflow strut 308 disposed between the roots 310 of the outflow strut 308 and the apex region 302.

FIG. 10 shows an exemplary positioning of a radially expanded prosthetic heart valve 320 comprising the frame 300 deployed within the aortic root 322 of the aorta 323. Alternatively, the prosthetic heart valve 320 could comprise the frame 370 of FIG. 9A instead of the frame 300. Similar to the prosthetic heart valve 100 of FIG. 1, the prosthetic heart valve 320 can comprise a plurality of leaflets 112 mounted within an interior of the frame 300 and an outer skirt 106 disposed around an outer surface of the frame 300.

The aortic root 322 includes the right coronary ostia 324 into the right coronary artery 328 and the left coronary ostia 326 into the left coronary artery 330 (FIG. 10). Further, the aortic root 322 is defined between the native aortic valve annulus 332 and the sinotubular junction (STJ) 334 (the junction between the sinus 336 and the ascending aorta 338). As shown in FIG. 10, the prosthetic heart valve 320 can be deployed within the aortic root 322 such that the outwardly bent apex regions 302 contact the sinus wall 340 at or adjacent to the sinus ceiling 342, below the STJ 334. In some examples, the apex regions 302 can contact the sinus wall 340 at the STJ 334. As shown in in FIG. 10, the sinus ceiling 342 can be angled or inclined relative to a central longitudinal axis of the aorta 323 (and the central longitudinal axis of the prosthetic heart valve 320). Further, the sinus wall can funnel or taper from the narrower STJ to the right and left coronary ostia 324 and 326.

In some examples, the angle 304 of the apex regions 302 can be specified such that the apex regions 302 abut the inclined sinus ceiling 342 at an angle of approximately 90 degrees. For example, a longitudinal axis of the apex regions 302 (extending through an apex of each apex region) can be perpendicular to the sinus ceiling 342. Said another way, a plane parallel to the apex of the apex region 302 can be approximately parallel to a plane of the sinus ceiling 342 (at a location where the apex region 302 abuts the sinus ceiling). As a result, a distance 345 between the apex regions 302 (and the outflow end of the frame) and the edge portions of the host leaflets (e.g., of a previously implanted prosthetic heart valve within which the prosthetic heart valve 320 is implanted, or of the native aortic valve) can be maximized, thereby maximizing a size of a window or opening 347 through the frame 300 (e.g., for a coronary access device or catheter to pass through). For example, though not shown in FIG. 10, the outflow edge portions of the leaflets of a host prosthetic heart valve (previously implanted in the native aortic valve) can be disposed at a same level as, or slightly higher or lower than, the outflow edge portions of the leaflets 112 of the newly implanted prosthetic heart valve 320.

Additionally, the specified angle 304 of the apex regions 302 can result in a relatively straight portion of the frame 300 (between roots 310 of the outflow struts 308 and the inflow end 108 of the frame 300, or the intermediate portion 312 of the frame 300) being spaced away from the sinus wall 340 by a lateral spacing 346 that can be in a range of 0-10 mm, 0.5-6 mm, or 4-6 mm, thereby providing clearance between an outer surface of the frame 300 and the sinus wall 340. In some instances, the angle 304 can be a non-zero angle that is greater than 10 degrees, in a range of 10 to 60 degrees, or in a range of 15 to 50 degrees.

As shown in FIG. 10, the outwardly bent apex regions 302 can maintain the prosthetic heart valve 320 axially below the STJ 334 and prevent it from migrating into the ascending aorta 338. This positioning can result in the leaflets 112 of the prosthetic heart valve 320 being spaced farther away from the STJ 334 and the sinus ceiling 342 (shown by spacing 344 in FIG. 10), thereby reducing a likelihood of blocking access to the right and left coronary ostia 324, 326 following a valve-in-valve procedure.

As introduced above, the apex regions 302 can be bent radially outward during radial expansion with the delivery apparatus, such as during radially expanding the prosthetic heart valve by inflating the balloon 350 of the delivery apparatus 352.

In some examples, the balloon 350 can be configured to have a non-uniform compliance with one or more sections (or segments) that are configured to apply an increased radially outward pressure against the frame 300 relative to remaining sections of the balloon 350. For example, FIG. 8 depicts the balloon 350 comprising a first or reduced compliance section 354 (or segment) and a second or increased compliance section 356 (or segment). In some examples, the reduced compliance section 354 can be referred to as a non-compliant section that is configured to expand (inflate) to a specified diameter and exert a relatively high radially outward pressure against the frame 300. As a result, the reduced compliance section 354 can be configured to exert a radially outward pressure that results in the radially outward bending of the apex regions 302, as shown in FIG. 8 (and FIGS. 5, 6B, and 10). In some instances, the reduced compliance section 354 can comprise polyester or nylon. A material of the reduced compliance section 354 can be selected to achieve the specified amount of outward bending of the apex regions 302 (e.g., the desired angle 304).

In some examples, the increased compliance section 356 can be referred to as a semi-compliant section that is configured to expand to a specified range of diameters (e.g., it is compliant with a limit to its expansion diameter, as depicted by dashed lines 358 in FIG. 8). As a result, the increased compliance section 356 can be configured to radially expand the frame 300 to its radially expanded configuration (e.g., the expansion diameter depicted by dashed lines 358). In some instances, the increased compliance section 356 can comprise Pebax or a higher-durometer polyurethane. Thus, the radially outward pressure applied by the reduced compliance section 354 is greater than the radially outward pressure applied by the increased compliance section 356.

In this way, the sections of the balloon 350 can be specifically configured to achieve the outward bending of the apex regions 302, while expanding a remainder of the frame 300 to a more cylindrical shape (as shown in FIGS. 6B and 10) during radial expansion of the frame 300 via inflation of the balloon 350.

In alternate examples, instead of a varying compliance balloon, the apex regions 302 can be configured to bend radially outward during radial expansion with a balloon 360 having a more uniform compliance (or a single compliance), such as a compliant or semi-compliant balloon, as shown in FIG. 9B. In such examples, the outflow struts 308 can comprise a thinned portion 362 (or neck region) that is narrower in a radial direction compared to a remaining portion of the outflow strut 308 (and other struts of the frame). For example, FIG. 9A illustrates a cross-section of another exemplary frame 370 that may be the same as frame 300, except for the presence of thinned portions 362 in the outflow struts 308. In some instances, as shown in FIG. 9A, the thinned portions 362 can be disposed at the roots 310 (on both ends) of the outflow struts 308. As such, when the balloon 360 is expanded, as depicted in FIG. 9B, the apex regions 302 are bent radially outward while a remainder of the frame 370 (e.g., a relatively straight portion 364 disposed between the roots 310 of the outflow struts 308 and the inflow end 108 of the frame 370) is expanded to its target expanded diameter 366. As shown in FIG. 9B, the target expanded diameter 366 is smaller than the expanded diameter 368 of the frame 370 at the apices of the apex regions 302. In this way, the thinned portions 362 at the roots 310 of the outflow struts 308 create weaker or more flexible regions in the outflow struts 308 that enable the outflow struts 308 and apex regions 302 to bend radially outward with a same radially outward pressure that is applied by the balloon 360 to the relatively straight portion 364 of the frame 370.

FIG. 11 shows an exemplary method 400 for implanting a prosthetic heart valve within an aortic root of a heart with radially outwardly bent apex regions or apices at an outflow end that hold the prosthetic heart valve in position at or below a sinotubular junction (and/or sinus ceiling). In alternate examples, method 400 can be used to implant a prosthetic heart valve in an alternate location, such as within an alternate heart valve annulus or vessel, with radially outwardly bent apex regions or apices. The prosthetic heart valve described below can be the prosthetic heart valve 320 comprising the frame 300 or a similar prosthetic heart valve comprising the frame 370. Further, the delivery apparatus described below can be any of the delivery apparatuses described herein, such as the delivery apparatus 200 of FIG. 4 or the delivery apparatus 352 of FIG. 7.

The method 400 begins at 402 by advancing a prosthetic heart valve radially compressed around a distal end portion of a delivery apparatus to an implantation site using the delivery apparatus. The distal end portion of the delivery apparatus can include an inflatable balloon (e.g., the balloon 350 of FIGS. 7 and 8 or the balloon 360 of FIG. 9B). The prosthetic heart valve can be positioned around the balloon. In some examples, the implantation site is a native aortic valve annulus and an aortic root of a heart, the aortic root including the right and left coronary ostia and defined between the native aortic valve annulus and the sinotubular junction (STJ).

At 404, the method 400 includes inflating the balloon of the delivery apparatus at the implantation site to radially expand and implant the prosthetic heart valve, where the inflating the balloon includes applying a radially outward pressure to a frame of the prosthetic heart valve with the balloon such that apex regions disposed at an outflow end of the frame are bent radially outward at an angle from remaining struts of the frame and relative to a longitudinal axis of the remaining struts that extends from an inflow end of the frame toward the outflow end. The angle can be a non-zero angle, such as the angle 304 described above with reference to FIG. 6B. Thus, the apex regions at the outflow end can be angled radially outward relative to the longitudinal axis (e.g., the longitudinal axis 306 shown in FIGS. 5-6B and 9B) while the remaining struts of the frame (such as the axially extending struts to which the outflow struts are connected) are aligned with the longitudinal axis.

In some examples, the balloon is a varying compliance balloon (e.g., the balloon 350 of FIG. 8) and the method at 404 can include applying a first radially outward pressure at the apex regions at the outflow end of the frame (e.g., the apex regions 302) with a non-compliant segment of the balloon (e.g., the reduced compliance section 354) and a second radially outward pressure at the remaining struts of the frame with a semi-compliant segment of the balloon (e.g., the increased compliance section 356), where the first radially outward pressure is larger than the second radially outward pressure. In alternate examples, instead of the non-compliant and semi-compliant segments, the balloon can comprise a first segment and a second segment, the first segment having a lower compliance and configured to exert the larger, first radially outward pressure against the outflow apex regions.

In alternate examples, the balloon has a relatively constant compliance throughout (e.g., the balloon 360 of FIG. 9B) and the method at 404 can include pivoting and bending the apex regions at the outflow end radially outward at the angle from roots of the outflow struts of the frame (which include the apex regions). The roots of the outflow struts can have a reduced thickness relative to a remainder of the outflow struts and the axially extending struts to which the outflow struts are connected via the roots. As such, the roots can form a neck region and pivot point for the outflow struts to bend radially outward from in response the radially outward pressure applied by the balloon.

The inflating the balloon at 404 can further include radially expanding the remaining struts of the frame to a specified expansion diameter that is relatively uniform along the longitudinal axis of the remaining struts. In some examples, the longitudinal axis of the remaining struts can be parallel to a longitudinal axis of the frame.

In still other examples, the balloon can be a varying compliance balloon and the roots of the outflow struts of the frame can have the thinned portions, thereby enabling the apex regions to bend radially outward from the roots of the outflow struts during expansion of the balloon.

At 406, the method 400 includes implanting the radially expanded prosthetic heart valve at a native valve annulus, within an aortic root of a heart, with at least a portion of the radially outwardly bent apex regions abutting a wall of the aortic root, at or below the STJ. For example, as shown in FIG. 10 at least a portion of the outflow apex regions 302 can contact and/or be disposed against the wall of the aortic root (e.g., the sinus wall 340). As a result, the apex regions can help to hold the prosthetic heart valve in place within the aortic root and prevent the prosthetic heart valve from migrating closer to the STJ and into the ascending aorta. Additionally or alternatively, the method at 406 can include implanting the radially expanded prosthetic heart valve at the native valve annulus, within the aortic root, with at least the portion of the radially outwardly bent apex regions abutting a wall of the aortic root such that a longitudinal axis of the portion of the radially outwardly bent apex regions is perpendicular to the wall of the aortic root (e.g., the angled sinus ceiling wall)

In alternate examples, the method at 406 can include implanting the radially expanded prosthetic heart valve (or prosthetic device) at a different native valve annulus, or within a vessel, with at least a portion of the radially outwardly bent apex regions (at the outflow end and/or an inflow end of the prosthetic heart valve) abutting a wall of the annulus or vessel, thereby helping to anchor the prosthetic heart valve within the annulus or vessel.

As introduced above, by configuring a frame of a prosthetic heart valve with apices or apex regions (e.g., apex regions 302) that are configured to bend radially outward during radial expansion of the frame with a delivery apparatus at the implantation site, the prosthetic heart valve can be positioned within the aortic root at or below the STJ or sinus ceiling, thereby spacing the outflow ends of the leaflets of the prosthetic heart valve further away from the STJ and providing a larger space for access to the coronary arteries through the frame of the prosthetic heart valve (for both an initial prosthetic heart valve implantation within a native heart valve and a valve-in-valve procedure). For example, by configuring the apex regions at the outflow end of the frame to bend radially outward and abut the angled sinus ceiling, a distance between the outflow end of the frame and the host valve leaflets (of the native heart valve and or a previously implanted prosthetic valve) can be maximized, thereby increasing a size of the open windows through the frame of the newly prosthetic heart valve for a catheter or other coronary access device to access the coronary arteries. As a result, a serviceability of the implanted prosthetic heart valve (during future interventions) can be increased.

Further, such alignment within the aortic root can be stabilized by the outwardly bent apex regions' interaction with the sinus wall, thereby structurally holding the prosthetic heart valve in position within the aortic root such that it does not migrate into the ascending aorta during operation (opening and closing of the leaflets) of the prosthetic heart valve. Additionally, the outwardly bent apex regions at the outflow end of the prosthetic heart valve can provide better resistance to cyclic force on commissure (FOC) loading. For example, the commissures of the prosthetic heart valve can experience cyclic force components (in the radial and axial directions) during the repeated opening and closing of the leaflets during valve operation. The radial taper of the outwardly bent apex regions at the outflow end, proximate to the commissures, can reduce these forces, thereby increasing and overall durability of the prosthetic heart valve.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parastemal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Simulation

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: an annular frame comprising a plurality of interconnected struts, the plurality of interconnected struts arranged in a plurality of rows of struts, including a first row of first struts defining a first end of the frame, each first strut comprising an apex region disposed between two angled strut portions of the first strut, wherein the frame is radially expandable from a radially compressed, delivery configuration to a radially expanded, deployed configuration, and wherein each apex region is configured to bend radially outward from a first configuration to a second configuration where the apex region is bent radially outward at an angle relative to remaining struts in rows other than the first row of first struts, the apex region being in the first configuration when the frame is in the delivery configuration and in the second configuration when the frame is in the deployed configuration.

Example 2. The prosthetic heart valve of any example herein, particularly example 1, wherein in the first configuration, the apex region is axially aligned with the remaining struts.

Example 3. The prosthetic heart valve of any example herein, particularly either example 1 or example 2, wherein the angle is defined between a longitudinal axis extending along the remaining struts from a second end of the frame to the first struts, and wherein the angle is greater than 10 degrees.

Example 4. The prosthetic heart valve of any example herein, particularly any one of examples 1-3, wherein the plurality of interconnected struts comprises a second row of second struts defining a second end of the frame, each second strut comprising an apex region disposed between two angled strut portions of the second strut, and wherein each apex region at the second end remains in a same unbent configuration in both the delivery configuration and the deployed configuration of the frame.

Example 5. The prosthetic heart valve of any example herein, particularly example 4, wherein each first strut forms an edge of a cell of a first row of cells disposed at the first end of the frame, wherein each second strut forms an edge of a cell of a second row of cells disposed at the second end of the frame, and wherein the cell of the first row of cells has a longer axial length, relative to a central longitudinal axis of the frame, than the cell of the second row of cells.

Example 6. The prosthetic heart valve of any example herein, particularly any one of examples 1-5, wherein the first end of the frame is an outflow end, and the second end of the frame is an inflow end.

Example 7. The prosthetic heart valve of any example herein, particularly any one of examples 1-6, wherein for each first strut, the apex region is configured to bend radially outward from a root of each angled strut portion.

Example 8. The prosthetic heart valve of any example herein, particularly example 7, wherein the root of each angled strut portion connects to an axially extending strut of the plurality of interconnected struts of the frame.

Example 9. The prosthetic heart valve of any example herein, particularly example 8, wherein the root of each angled strut portion has a first thickness defined in a radial direction that is smaller than a second thickness of a remaining portion of the angled strut portion and the axially extending strut to which it connects.

Example 10. The prosthetic heart valve of any example herein, particularly any one of examples 1-9, wherein the apex region of each first strut curves between the two angled strut portions and has a narrowed width relative to a width of the two angled strut portions.

Example 11. The prosthetic heart valve of any example herein, particularly any one of examples 1-10, wherein the apex region forms an angle between the two angled strut portions that is greater than 120 degrees.

Example 12. The prosthetic heart valve of any example herein, particularly any one of examples 1-11, wherein the apex region comprises a curved, axially facing outer surface that is continuous with axially facing outer surfaces of the two angled strut portions and an axially facing inner depression, the inner depression depressed toward the curved outer surface from axially facing inner surfaces of the two angled strut portions.

Example 13. The prosthetic heart valve of any example herein, particularly any one of examples 1-12, wherein the plurality of interconnected struts define a plurality of rows of cells arranged between the first end and a second end of the frame, and further comprising a plurality of leaflets secured to the frame and a plurality of commissure windows formed by struts of the plurality of interconnected struts forming cells of a first row of cells of the plurality of rows of cells, the first row of cells disposed at the first end of the frame, and wherein each commissure window is configured to receive commissure tabs of two adjacent leaflets of the plurality of leaflets.

Example 14. A prosthetic heart valve comprising: a radially expandable and compressible annular frame comprising a plurality of interconnected struts defining a plurality of rows of cells arranged between an inflow end and an outflow end of the frame, the plurality of interconnected struts comprising a plurality of outflow struts defining the outflow end, wherein each outflow strut comprises two angled strut portions interconnected by an apex region, and wherein each outflow strut is configured to bend radially outward in response to a radially outward force applied thereto during radial expansion of the frame and deployment of the prosthetic heart valve such that the apex region of each outflow strut extends radially outward at an angle relative to a longitudinal axis of remaining struts of the plurality of interconnected struts when the frame is in a radially expanded and deployed configuration, wherein the remaining struts are disposed between the outflow struts and the inflow end of the frame.

Example 15. The prosthetic heart valve of any example herein, particularly example 14, wherein each outflow strut is configured to bend radially outward from opposite ends of the outflow strut that connect to axially extending struts of the plurality of interconnected struts.

Example 16. The prosthetic heart valve of any example herein, particularly example 15, wherein the axially extending struts form axial sides of a first row of cells of the plurality of rows of cells.

Example 17. The prosthetic heart valve of any example herein, particularly example 16, wherein each cell of the first row of cells has a first axial length and each cell of an adjacent, second row of cells of the plurality of rows of cells has a second axial length, the first axial length longer than the second axial length.

Example 18. The prosthetic heart valve of any example herein, particularly any one of examples 15-17, wherein the opposite ends of the outflow struts that connect to the axially extending struts form neck regions having a first thickness that is smaller than a second thickness of a remaining portion of the angled strut portions of the outflow struts, the first thickness and the second thickness defined in a radial direction of the frame.

Example 19. The prosthetic heart valve of any example herein, particularly any one of examples 14-18, wherein the plurality of interconnected struts forms a plurality of circumferentially extending rows of struts including a first row of struts formed by the plurality of outflow struts and a second row of struts formed by a plurality of inflow struts defining the inflow end of the frame, wherein each inflow strut comprises an apex region disposed between two angled strut portions of the inflow strut, and wherein each apex region at the inflow end remains in a same unbent configuration in both the radially expanded and deployed configuration of the frame and a radially compressed, delivery configuration of the frame, prior to radial expansion.

Example 20. The prosthetic heart valve of any example herein, particularly any one of examples 14-19, wherein the angle is in a range of 10 to 60 degrees.

Example 21. The prosthetic heart valve of any example herein, particularly any one of examples 14-20, wherein each outflow strut is configured to transition from a first configuration to a second configuration in response to the radially outward force applied thereto during radial expansion of the frame and deployment of the prosthetic heart valve, wherein in the second configuration the apex region of each outflow strut extends radially outward at the angle relative to the longitudinal axis of the remaining struts, and wherein in the first configuration the apex region of each outflow strut is axially aligned with the longitudinal axis of the remaining struts.

Example 22. The prosthetic heart valve of any example herein, particularly any one of examples 14-21, wherein the apex region of each outflow strut curves between the two angled strut portions and has a narrowed width relative to a width of the two angled strut portions.

Example 23. The prosthetic heart valve of any example herein, particularly any one of examples 14-22, wherein the apex region forms an angle between the two angled strut portions that is greater than 120 degrees.

Example 24. The prosthetic heart valve of any example herein, particularly any one of examples 14-23, wherein the apex region comprises a curved, axially facing outer surface that is continuous with axially facing outer surfaces of the two angled strut portions and an axially facing inner depression, the inner depression depressed toward the curved outer surface from axially facing inner surfaces of the two angled strut portions.

Example 25. The prosthetic heart valve of any example herein, particularly any one of examples 14-24, further comprising a plurality of leaflets secured to the frame and a plurality of commissure windows formed by struts of the plurality of interconnected struts forming cells of a first row of cells of the plurality of rows of cells, the first row of cells disposed at the outflow end of the frame, and wherein each commissure window is configured to receive commissure tabs of two adjacent leaflets of the plurality of leaflets.

Example 26. A method comprising: advancing a prosthetic heart valve that is radially compressed around a distal end portion of a delivery apparatus to an implantation site using the delivery apparatus, wherein the distal end portion of the delivery apparatus includes an inflatable balloon; and inflating the balloon of the delivery apparatus at the implantation site to radially expand and implant the prosthetic heart valve, wherein the inflating the balloon includes applying a radially outward pressure to a frame of the prosthetic heart valve with the balloon such that apex regions disposed at an outflow end of the frame are bent radially outward at an angle from remaining struts of the frame and relative to a longitudinal axis of the remaining struts that extends from an inflow end of the frame toward the outflow end, wherein the frame comprises a plurality of interconnected struts arranged in a plurality of rows of struts, including a first row of outflow struts defining the outflow end of the frame and including the apex regions, and wherein the remaining struts are disposed in rows other than the first row of outflow struts.

Example 27. The method of any example herein, particularly example 26, wherein the angle is a non-zero angle, and wherein apex regions at the inflow end of the frame which are defined by a second row of inflow struts are not bent radially outward during inflating the balloon, relative to remaining struts that are disposed in rows other than the first row of outflow struts and the second row of inflow struts.

Example 28. The method of any example herein, particularly either example 26 or example 27, wherein each outflow strut comprises two angled strut portions interconnected by a respective apex region of the apex regions, and wherein each angled strut portion of each outflow strut connects to an axially extending strut of a plurality of axially extending struts of the plurality of interconnected struts at a root of the outflow strut.

Example 29. The method of any example herein, particularly example 28, wherein the inflating the balloon includes pivoting and bending the apex regions at the outflow end radially outward at the angle from roots of the outflow struts.

Example 30. The method of any example herein, particularly example 29, wherein the roots of the outflow struts have a reduced thickness, in a radial direction of the frame, relative to a remaining portion of the outflow struts and the axially extending struts.

Example 31. The method of any example herein, particularly any one of examples 28-30, wherein each apex region has a curved axially outward facing surface that curves between the two angled strut portions and a first width that is thinner than a second width of the two angled strut portions.

Example 32. The method of any example herein, particularly any one of examples 26-29, wherein the balloon is a varying compliance balloon, wherein the inflating the balloon includes applying a first radially outward pressure at the apex regions at the outflow end of the frame with a first segment of the balloon and applying a second radially outward pressure at the remaining struts of the frame with a second segment of the balloon, and wherein the first radially outward pressure is larger than the second radially outward pressure.

Example 33. The method of any example herein, particularly example 32, wherein the first segment is a non-compliant segment of the balloon, and wherein the second segment is a semi-compliant segment of the balloon.

Example 34. The method of any example herein, particularly any one of examples 26-33, wherein inflating the balloon further includes radially expanding the remaining struts of the frame to a specified expansion diameter that is uniform along the longitudinal axis of the remaining struts.

Example 35. The method of any example herein, particularly any one of examples 26-34, wherein the longitudinal axis of the remaining struts is parallel to a central longitudinal axis of the frame.

Example 36. The method of any example herein, particularly any one of examples 26-35, wherein the implantation site is a native aortic valve annulus and an aortic root of a heart, the aortic root defined between the native aortic valve annulus and a sinotubular junction.

Example 37. The method of any example herein, particularly any one of examples 26-36, further comprising implanting the prosthetic heart valve into a native valve or a previously implanted prosthetic heart valve.

Example 38. A method comprising: radially expanding a prosthetic heart valve to a radially expanded configuration at a native valve annulus within an aortic root of a heart by expanding an inflatable balloon of a delivery apparatus around which the prosthetic heart valve is mounted, the prosthetic heart valve comprising a frame comprising a plurality of interconnected struts, the plurality of interconnected struts arranged in a plurality of circumferentially extending rows of struts including a first row of outflow struts defining an outflow end of the frame and a second row of inflow struts defining an inflow end of the frame; and in response to expanding the inflatable balloon, bending the outflow struts radially outward from struts of the frame to which the outflow struts are connected such that, after radially expanding the prosthetic heart valve to the radially expanded configuration, an apex region of each outflow strut is positioned radially outward relative to remaining struts of the frame that are disposed in rows of struts other than the first row of outflow struts and, for at least a portion of the outflow struts, the apex region abuts a wall of the aortic root at or below a sinotubular junction.

Example 39. The method of any example herein, particularly example 38, wherein the plurality of interconnected struts includes a plurality of axially extending struts that are spaced circumferentially apart around the frame and which are connected to roots of the outflow struts, and wherein the outflow struts are bent radially outward from the roots of the outflow struts.

Example 40. The method of any example herein, particularly example 39, wherein the roots of the outflow struts have a reduced thickness relative to a remaining portion of the outflow struts and the plurality of axially extending struts.

Example 41. The method of any example herein, particularly any one of examples 38-40, wherein the bending the outflow struts includes bending the outflow struts radially outward at a non-zero angle relative to a longitudinal axis of the remaining struts of the frame.

Example 42. The method of any example herein, particularly example 41, wherein the angle is greater than 10 degrees.

Example 43. The method of any example herein, particularly any one of examples 38-42, wherein the radially expanding the prosthetic heart valve includes radially expanding the prosthetic heart valve such that, after radially expanding to the prosthetic heart valve to the radially expanded configuration, an apex region of each inflow strut of the second row of inflow struts is axially aligned with remaining struts of the frame that are disposed in rows of struts other than the first row of outflow struts and the second row of inflow struts.

Example 44. The method of any example herein, particularly any one of examples 38-43, wherein each outflow struts comprises two angled struts portions interconnected by the apex region of the outflow strut, and wherein the apex region of each outflow strut has a curved axially outward facing surface that curves between the two angled strut portions and a first width that is thinner than a second width of the two angled strut portions.

Example 45. The method of any example herein, particularly any one of examples 38-44, wherein the balloon is a varying compliance balloon, and wherein, in response to expanding the inflatable balloon, bending the outflow struts radially outward from struts of the frame to which the outflow struts are connected includes: applying a first radially outward pressure to the outflow struts with a first segment of the balloon; and applying a second radially outward pressure to the remaining struts of the frame with a second segment of the balloon, wherein the first radially outward pressure is larger than the second radially outward pressure.

Example 46. The method of any example herein, particularly example 45, wherein the second segment has a higher compliance than the first segment of the balloon.

Example 47. The method of any example herein, particularly any one of examples 38-46, wherein radially expanding the prosthetic heart valve to the radially expanded configuration includes radially expanding the remaining struts of the frame to a specified expansion diameter that is uniform along a longitudinal axis of the remaining struts.

Example 48. The method of any example herein, particularly any one of examples 38-47, further comprising implanting the prosthetic heart valve into a native valve or a previously implanted prosthetic heart valve within the aortic root, wherein the wall of the aortic root includes an angled sinus ceiling of the heart, and wherein for at least the portion of the outflow struts, the apex region abuts the angled sinus ceiling perpendicular to the angled sinus ceiling.

Example 49. A method comprising: applying a radially outward pressure with a balloon of a delivery apparatus to an inner surface of a frame of a prosthetic heart valve to radially expand the prosthetic heart valve and, during the applying the radially outward pressure, bending outflow struts defining an outflow end of the frame radially outward from roots of the outflow struts that connect to axially extending struts of the frame such that apex regions of the outflow struts extend radially outward at an angle relative to a longitudinal axis of the axially extending struts; and implanting the radially expanded prosthetic heart valve at a native valve annulus, within an aortic root of a heart, with at least a portion of the apex regions at the outflow end abutting a wall of the aortic root, below a sinotubular junction, while a remainder of the frame is spaced apart from the wall of the aortic root.

Example 50. The method of any example herein, particularly example 49, wherein the remainder of the frame extends from the roots of the outflow struts to an inflow end of the frame and includes the axially extending struts.

Example 51. The method of any example herein, particularly either example 49 or example 50, wherein the remainder of the frame is spaced apart from the wall of the aortic root in a radial direction such that there is a gap between the wall and an outer surface of the remainder of the frame.

Example 52. The method of any example herein, particularly any one of examples 49-51, wherein the angle is in a range of 10 to 60 degrees.

Example 53. The method of any example herein, particularly any one of examples 49-52, wherein applying the radially outward pressure with the balloon includes applying a first radially outward pressure to the outflow struts with a first segment of the balloon, the first segment having a first compliance, and applying a second radially outward pressure to the remainder of the frame with a second segment of the balloon, the second segment having a second compliance that is greater than the first compliance, and wherein the first radially outward pressure is larger than the second radially outward pressure.

Example 54. The method of any example herein, particularly any one of examples 49-52, wherein the roots of the outflow struts have a first thickness, defined in a radial direction of the frame, that is smaller than a second thickness of a remaining portion of the outflow struts and the axially extending struts.

Example 55. A prosthetic heart valve comprising: a radially expandable and compressible annular frame comprising a plurality of interconnected struts comprising a plurality of rows of struts including a first row of outflow struts defining an outflow end of the frame and a second row of struts connected to the first row of outflow struts, wherein each outflow strut comprises: two angled strut portions interconnected by an apex region, wherein an end of each angled strut portion that is disposed away from the apex region and connects to a respective strut of the second row of struts has a neck region that has a narrowed thickness in a radial direction relative to a remainder of the outflow strut and the respective strut of the second row of struts, and wherein a portion of the outflow strut including the apex region is configured to pivot and bend radially outward from the neck region of each angled strut portion relative to remaining struts in rows other than the first row of outflow struts, in response to a radially outwardly directed force applied to an inner surface of the frame.

Example 56. The prosthetic heart valve of any example herein, particularly example 55, wherein the second row of struts includes a plurality of axially extending struts that are spaced apart from one another around the frame, and wherein the plurality of axially extending struts extend between the first row of outflow struts and a third row of angled struts.

Example 57. The prosthetic heart valve of any example herein, particularly example 56, wherein the plurality of interconnected struts defines a plurality of rows of cells arranged between the outflow end and an inflow end of the frame, and wherein the first row of outflow struts, the plurality of axially extending struts, and the third row of angled struts form a first row of cells of the plurality of rows of cells.

Example 58. The prosthetic heart valve of any example herein, particularly example 57, further comprising a plurality of leaflets secured to the frame and a plurality of commissure windows formed by a portion of the axially extending struts, and wherein each commissure window is configured to receive commissure tabs of two adjacent leaflets of the plurality of leaflets.

Example 59. The prosthetic heart valve of any example herein, particularly any one of examples 55-58, wherein the radially outwardly directed force is applied to the inner surface of the frame during radial expansion of the frame and deployment of the prosthetic heart valve to a radially expanded configuration, and wherein in the radially expanded configuration the apex region of each outflow strut extends radially outward at an angle from a longitudinal axis of the remaining struts.

Example 60. The prosthetic heart valve of any example herein, particularly example 59, wherein the angle is greater than 10 degrees.

Example 61. The prosthetic heart valve of any example herein, particularly any one of examples 55-60, wherein the apex region of each outflow strut curves between the two angled strut portions and has a narrowed width relative to a width of the two angled strut portions.

Example 62. The prosthetic heart valve of any example herein, particularly any one of examples 55-61, wherein the apex region forms an angle between the two angled strut portions that is greater than 120 degrees.

Example 63. The prosthetic heart valve of any example herein, particularly any one of examples 55-62, wherein the apex region comprises a curved, axially facing outer surface that is continuous with axially facing outer surfaces of the two angled strut portions and an axially facing inner depression, the inner depression depressed toward the curved outer surface from axially facing inner surfaces of the two angled strut portions.

Example 64. An assembly comprising: an inflatable balloon disposed at a distal end portion of a delivery apparatus; and a prosthetic heart valve mounted around the balloon in a radially compressed configuration, wherein the prosthetic heart valve comprises: an annular frame comprising a plurality of interconnected struts, the plurality of interconnected struts arranged in a plurality of rows of struts, including a first row of outflow struts defining an outflow end of the frame, each outflow strut comprising an apex region disposed between two angled strut portions of the outflow strut, wherein each apex region is configured to bend radially outward from a first configuration to a second configuration as a result of expansion of the balloon, wherein in the second configuration the apex region is bent radially outward at an angle relative to remaining struts in rows other than the first row of first struts.

Example 65. The assembly of any example herein, particularly example 64, wherein for each outflow strut, the apex region is configured to bend radially outward from a root of each angled strut portion.

Example 66. The assembly of any example herein, particularly example 65, wherein the root of each angled strut portion connects to an axially extending strut of the plurality of interconnected struts of the frame.

Example 67. The assembly of any example herein, particularly example 66, wherein the root of each angled strut portion has a first thickness defined in a radial direction that is smaller than a second thickness of a remaining portion of the angled strut portion and the axially extending strut to which it connects.

Example 68. The assembly of any example herein, particularly any one of examples 64-67, wherein in the first configuration the apex region is axially aligned with the remaining struts, and wherein the apex region is in the first configuration when the prosthetic heart valve is in the radially compressed configuration.

Example 69. The assembly of any example herein, particularly any one of examples 64-68, wherein the apex region of each outflow strut curves between the two angled strut portions and has a narrowed width relative to a width of the two angled strut portions.

Example 70. The assembly of any example herein, particularly any one of examples 64-69, wherein the apex region forms an angle between the two angled strut portions that is greater than 120 degrees.

Example 71. The assembly of any example herein, particularly any one of examples 64-70, wherein the apex region comprises a curved, axially facing outer surface that is continuous with axially facing outer surfaces of the two angled strut portions and an axially facing inner depression, the inner depression depressed toward the curved outer surface from axially facing inner surfaces of the two angled strut portions.

Example 72. The assembly of any example herein, particularly any one of examples 64-71, wherein the plurality of interconnected struts includes a plurality of axially extending struts extending between the first row of outflow struts and a second row of angled struts, wherein the prosthetic heart valve further comprises a plurality of leaflets secured to the frame at commissure windows of the frame that are formed by a portion of the axially extending struts, and wherein each commissure window is configured to receive commissure tabs of two adjacent leaflets of the plurality of leaflets.

Example 73. A method comprising sterilizing the prosthetic heart valve, apparatus, and/or assembly of any example.

Example 74. A prosthetic heart valve of any one of examples 1-72, wherein the prosthetic heart valve is sterilized.

Example 75. The method of any example herein, particularly any one of examples 26-54, wherein the method can be performed on a living animal or on a simulation, such as on a cadaver, cadaver heart, anthropomorphic ghost, or simulator.

The features described herein with regard to any example can be combined with other features described in any one or more of the other examples, unless otherwise stated. For example, any one or more of the features of one frame can be combined with any one or more features of another frame. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
an annular frame comprising a plurality of interconnected struts, the plurality of interconnected struts arranged in a plurality of rows of struts, including a first row of first struts defining a first end of the frame, each first strut comprising an apex region disposed between two angled strut portions of the first strut, wherein the frame is radially expandable from a radially compressed, delivery configuration to a radially expanded, deployed configuration, and wherein each apex region is configured to bend radially outward from a first configuration to a second configuration where the apex region is bent radially outward at an angle relative to remaining struts in rows other than the first row of first struts, the apex region being in the first configuration when the frame is in the delivery configuration and in the second configuration when the frame is in the deployed configuration.

2. The prosthetic heart valve of claim 1, wherein in the first configuration, the apex region is axially aligned with the remaining struts.

3. The prosthetic heart valve of claim 1, wherein the angle is defined between a longitudinal axis extending along the remaining struts from a second end of the frame to the first struts, and wherein the angle is greater than 10 degrees.

4. The prosthetic heart valve claim 1, wherein the plurality of interconnected struts comprises a second row of second struts defining a second end of the frame, each second strut comprising an apex region disposed between two angled strut portions of the second strut, and wherein each apex region at the second end remains in a same unbent configuration in both the delivery configuration and the deployed configuration of the frame.

5. The prosthetic heart valve of claim 1, wherein the first end of the frame is an outflow end, and the second end of the frame is an inflow end.

6. The prosthetic heart valve of claim 1, wherein for each first strut, the apex region is configured to bend radially outward from a root of each angled strut portion, and wherein the root of each angled strut portion connects to an axially extending strut of the plurality of interconnected struts of the frame.

7. The prosthetic heart valve of claim 1, wherein the apex region of each first strut curves between the two angled strut portions and has a narrowed width relative to a width of the two angled strut portions.

8. A method comprising:
advancing a prosthetic heart valve that is radially compressed around a distal end portion of a delivery apparatus to an implantation site using the delivery apparatus, wherein the distal end portion of the delivery apparatus includes an inflatable balloon; and
inflating the balloon of the delivery apparatus at the implantation site to radially expand and implant the prosthetic heart valve, wherein the inflating the balloon includes applying a radially outward pressure to a frame of the prosthetic heart valve with the balloon such that apex regions disposed at an outflow end of the frame are bent radially outward at an angle from remaining struts of the frame and relative to a longitudinal axis of the remaining struts that extends from an inflow end of the frame toward the outflow end, wherein the frame comprises a plurality of interconnected struts arranged in a plurality of rows of struts, including a first row of outflow struts defining the outflow end of the frame and including the apex regions, and wherein the remaining struts are disposed in rows other than the first row of outflow struts.

9. The method of claim 8, wherein the angle is a non-zero angle, and wherein apex regions at the inflow end of the frame which are defined by a second row of inflow struts are not bent radially outward during inflating the balloon, relative to remaining struts that are disposed in rows other than the first row of outflow struts and the second row of inflow struts.

10. The method of claim 8, wherein each outflow strut comprises two angled strut portions interconnected by a respective apex region of the apex regions, and wherein each angled strut portion of each outflow strut connects to an axially extending strut of a plurality of axially extending struts of the plurality of interconnected struts at a root of the outflow strut.

11. The method of claim 10, wherein the inflating the balloon includes pivoting and bending the apex regions at the outflow end radially outward at the angle from roots of the outflow struts.

12. The method of claim 10, wherein each apex region has a curved axially outward facing surface that curves between the two angled strut portions and a first width that is thinner than a second width of the two angled strut portions.

13. The method of claim 8, wherein inflating the balloon further includes radially expanding the remaining struts of the frame to a specified expansion diameter that is uniform along the longitudinal axis of the remaining struts.

14. The method of claim 8, wherein the longitudinal axis of the remaining struts is parallel to a central longitudinal axis of the frame.

15. The method of claim 8, wherein the implantation site is a native aortic valve annulus and an aortic root of a heart, the aortic root defined between the native aortic valve annulus and a sinotubular junction.

16. The method of claim 8, further comprising implanting the prosthetic heart valve into a native valve or a previously implanted prosthetic heart valve.

17. An assembly comprising:
an inflatable balloon disposed at a distal end portion of a delivery apparatus; and
a prosthetic heart valve mounted around the balloon in a radially compressed configuration, wherein the prosthetic heart valve comprises:
an annular frame comprising a plurality of interconnected struts, the plurality of interconnected struts arranged in a plurality of rows of struts, including a first row of outflow struts defining an outflow end of the frame, each outflow strut comprising an apex region disposed between two angled strut portions of the outflow strut, wherein each apex region is configured to bend radially outward from a first configuration to a second configuration as a result of expansion of the balloon, wherein in the second configuration the apex region is bent radially outward at an angle relative to remaining struts in rows other than the first row of first struts.

18. The assembly of claim 17, wherein for each outflow strut, the apex region is configured to bend radially outward from a root of each angled strut portion.

19. The assembly of claim 18, wherein the root of each angled strut portion connects to an axially extending strut of the plurality of interconnected struts of the frame.

20. The assembly of claim 17, wherein the apex region comprises a curved, axially facing outer surface that is continuous with axially facing outer surfaces of the two angled strut portions and an axially facing inner depression, the inner depression depressed toward the curved outer surface from axially facing inner surfaces of the two angled strut portions.
